(19) United States
(12) Patent Application Publication
(10) Pub. No.: US 2016/0287740 A1
(43) Pub. Date: Oct. 6, 2016

(54) SUSPENSIONS AND MATERIALS COMPRISING COMPLEXES OF CHITIN NANOFIBRILS WITH METALS

(71) Applicant: MAVI SUD S.R.L., Aprilia (IT)

(72) Inventor: Pierfrancesco MORGANTI, Aprilia (IT)

(21) Appl. No.: 15/026,962

(22) PCT Filed: Oct. 2, 2014
(86) PCT No.: PCT/IB2014/065015

§ 371 Date: Apr. 1, 2016

(30) Foreign Application Priority Data
Oct. 2, 2013 (IT) RM2013A000535

(51) Int. Cl. A61L 15/28; A61L 27/54; A61L 27/20

(52) U.S. Cl. CPC A61L 15/28; A61L 27/20; A61L 27/54; A61L 2300/102; A61L 2300/104; A61L 2300/404; A61L 2400/12

(57) ABSTRACT

The present invention refers to an aqueous suspension comprising chitin nanofibrils in a complex with at least one metal selected from the group Ag, Cu and Bi and at least one thickener, as well as to a process for the preparation thereof. Moreover, the present invention provides a process for the manufacturing of biomedical materials from said suspension, and biomedical articles based on said materials.

The present invention refers to an aqueous suspension comprising chitin nanofibrils in a complex with metals and at least one thickener, and to biomedical materials and biomedical articles obtained from said suspensions. Moreover, the present invention provides a process for the preparation of such suspensions, biomedical materials and biomedical articles based on said materials.

### STATE OF THE PRIOR ART

In recent years, many efforts have been made for the development of materials having medical and biological purposes, in particular for the development of polymeric products suitable for tissue regeneration, cell technologies and organ transplantation. Fundamental characteristics that such materials must possess are biocompatibility and biodegradability, referred both to constituent polymers and products of their decomposition. In particular, polyglycols, polyacids, polylactones, polysaccharides and other natural and synthetic polymers are used for manufacturing bio-reabsorbable matrices (Langer R., Tirrell D. A. Designing materials for biology and medicine//Nature, 2004. Vol. 428,6982, pp. 487-492).

Recently, as matrices for biomedical applications, pellicles, tissues formed by irregular weaves of micro/nanofibres (non-woven tissues) and porous materials are used, based above all on the use of the natural polysaccharide chitin (CN) (Pillai C. K. S., Paul W., Sharma C. P. Chitin and chitosan polymers: Chemistry, solubility and fiber formation//Progress in Polymer Science. 2009. Vol. 34. P. 641-678). The utilization of said natural polymer increases every year owing to the non-toxic nature of this polysaccharide which is obtained from fish and crustacean processing waste, without depleting the environment of precious raw materials.

In particular, the N-deacetylated derivative of chitin, i.e. chitosan (CS), proved to be one among the most promising polymers due to its interesting properties, such as the absence of cytotoxicity, the easy bioabsorption, and the eco-friendliness of the manufacturing processes, which fostered its use in the biomedical field. However, due to the high hydrophilic nature, CS-based articles are unstable, fragile, scarcely elastic and substantially rigid in a humid state.

Object of the present invention is to develop materials based on chitin in crystalline form having improved characteristics with respect to materials known for use in biomedical engineering, in particular for the preparation of articles that be capable of fostering more effectively cell growth and tissue regeneration.

### SUMMARY OF THE INVENTION

The present invention is based on the discovery that suspensions of chitin nanofibrils with at least one metal selected from the group Ag, Cu and Bi and containing at least one thickener represent starting products suitable for obtaining materials and articles having the characteristics required for the biomedical uses described herein.

In particular, such suspensions enable the manufacturing, by casting or electrospinning techniques, of materials or articles suitable to foster cell growth and/or tissue regeneration, effectively eliminating or reducing the incompatibilities and the side effects that are observed with known materials/products and used for the same purpose.

The suspension of the invention, as well as the materials produced therewith, is characterized in that it has fungicidal/fungistatic, bactericidal/bacteriostatic properties due to the presence of metals such as Ag, Cu and/or Bi in a complex with chitin nanofibrils.

The materials of the invention are characterized in that they have mechanical properties, such as, e.g., ultimate tensile strength, making them particularly suitable for applications in the biomedical field.

In addition, the tridimensional architecture of the materials obtained by casting or electrospinning plays a significant role in fostering cell regeneration, proliferation and/or differentiation, as in its own structure it has spaces sufficiently wide to foster such cellular processes.

In particular, the materials, and the biomedical articles obtained therewith, are capable of fostering cell adhesion, formation of cell-cell junctions, sending of intercellular messages.

It follows that the materials and the articles described herein are suitable for use in the biomedical field.

Specifically, such materials and articles proved particularly advantageous when used as medications, for instance, of the bandage, plaster, gauze kind, etc. In fact, they allow to: (1) accelerate the cicatrization process thanks to the presence of chitin; (2) protect tissue damaged by exposure to environmental contaminants; (3) prevent or slow down bacterial attachment, thanks to the presence of nanofibrils in a complex with metals such as silver, copper and/or bismuth, thereby enabling, e.g., to leave gaps between a medication and the next one, with a remarkable reduction of patient's discomfort; (4) prevent the appearance of fungal infections, such as candida, on damaged skin, e.g., in patients subjected to antibiotic therapy; (5) be well-tolerated by patients, as said materials have a high biocompatibility and do not tend to join with the underlying tissues.

Therefore, object of the present invention are:
aqueous suspension comprising chitin nanofibrils in a complex with at least one metal selected from the group Ag, Cu and Bi and at least one thickener;
process for the preparation of the suspension of the invention;
process for the preparation of biomedical materials, characterized in that it comprises one or more steps wherein the suspension of the invention is transformed into a solid fine layer;
biomedical material in form of non-woven tissue, film, sheet, pellicle obtainable by casting and/or electrospinning of a suspension of the invention;
biomedical articles comprising the material of the invention.

Other advantages and features of the present invention will be made apparent in the following detailed description.

### DESCRIPTION OF THE FIGURES

FIGS. 1-3 show images, obtained with a scanning electron microscope (SEM), of a non-woven tissue made up from an embodiment of the suspension of the invention comprising chitin nanofibrils in a complex with metallic silver.

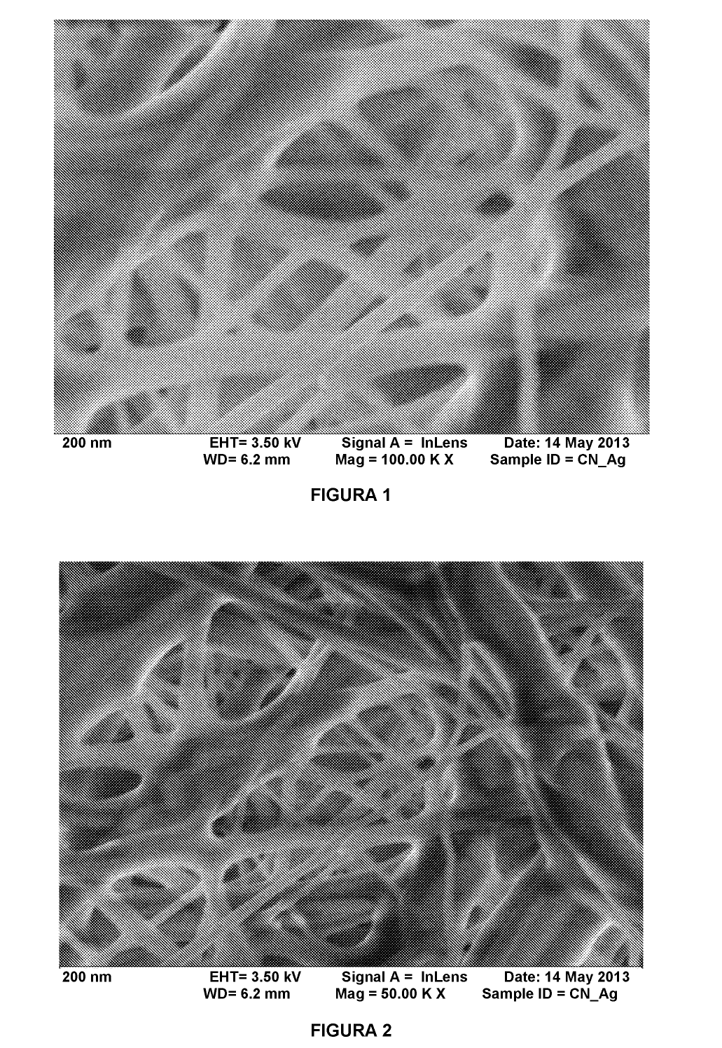

FIG. 4 shows an image, obtained with a scanning electron microscope (SEM), of an embodiment of a film made with the suspension of the invention comprising chitin nanofibrils and Cu.

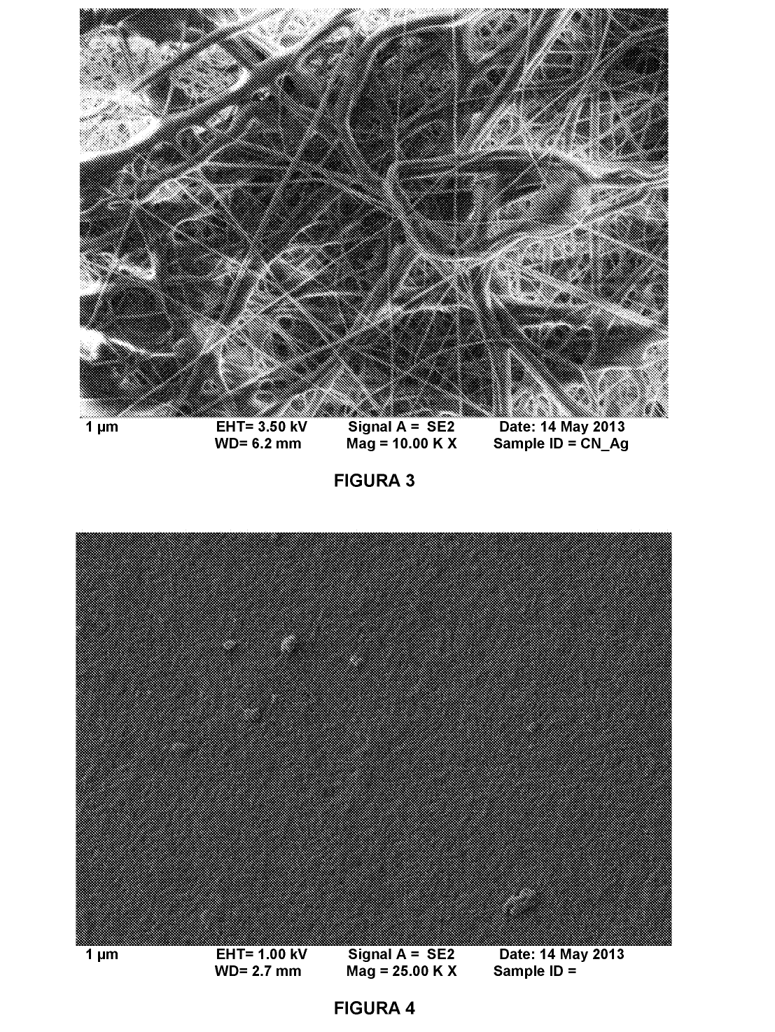

FIG. 5 shows an image, obtained with a scanning electron microscope (SEM), of chitin nanofibrils in a complex with silver (CN-Ag).

FIGS. 6A and 6B depict images related to an embodiment of a film according to the present invention. In particular, said figures show the film in a laid form (FIG. 6A) and in a folded form (FIG. 6B).

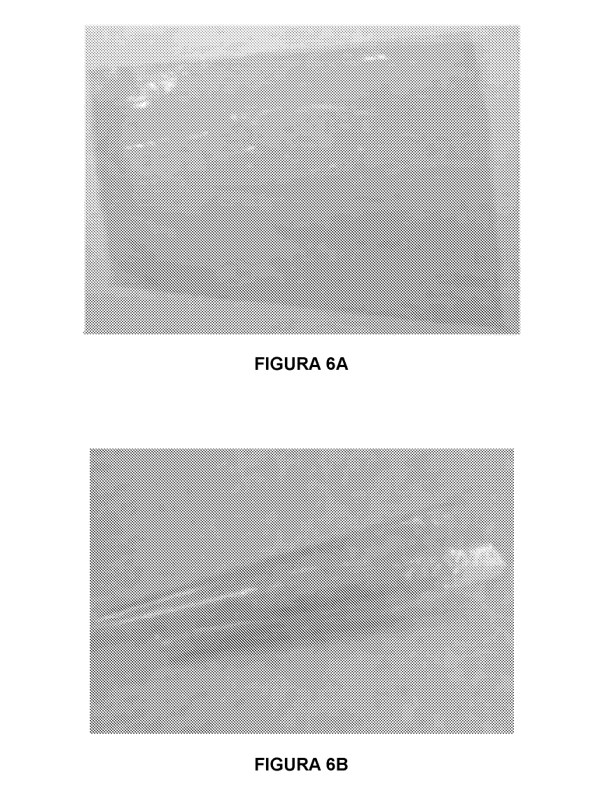

### DETAILED DESCRIPTION OF THE INVENTION

Hereinafter, a detailed description of the various objects of the present invention is reported.

Nanofibrils in a Complex with Metals.

For the purposes of the present invention, with the wording “chitin nanofibrils in a complex” or “chitin nanocrystals in a complex” it is meant chitin in its known form of nanometric fibril of a thickness/diameter ranging between 2.8-25 nm, average of 8 nm, and of about 200 nm of length (see WO-A-2006/048829) having on their surface one or more metals, and in particular selected from silver (Ag), copper (Cu), and bismuth (Bi), bonded by weak bonds (e.g., van der Waals bonds) and/or strong covalent-type bonds. In particular, the above metals can form a complex with chitin nanofibrils in any one of the respective oxidation states or in metallic form. Therefore, by way of example, silver can form a complex with chitin nanofibrils in its elementary metallic form (oxidation state 0); bismuth can form a complex with chitin nanofibrils in its oxidation state +2 or in its metallic form at an oxidation state 0; copper can form a complex with chitin nanofibrils in its oxidation state +1, +2, or in its metallic form (oxidation state 0).

In one embodiment of the invention, the chitin nanofibrils are in a complex with metals Ag, Cu and/or Bi in metallic form (oxidation state 0).

Process for the Preparation of Chitin Nanofibrils in a Complex with Metals

Hereinafter, also a process for the preparation of complexes of chitin nanofibrils with metals is described. Said process comprises at least one step of reduction, in aqueous solution, of metal ions in the presence of chitin nanofibrils.

In one embodiment of such process, the ions to be reduced are Ag, Cu and/or Bi ions, even though other metals with the same pharmacological activity may be used.

The metal ions to be reduced are present in the aqueous suspension in any one of their oxidation states.

Therefore, by way of example, silver can be present in the solution in the oxidation state +1; bismuth in the oxidation state +3 and/or +5; copper can be present in the solution in the oxidation state +1, +2, +3 and/or +4.

Metals are used in the aqueous solution in form of salts, such as, e.g., nitrites and/or sulfates.

In a preferred embodiment of the invention, the ions are selected from the group: silver nitrate or sulfate; copper nitrate or sulfate; bismuth nitrate or sulfate.

In the aqueous suspension, the ratio between the concentration of chitin nanofibrils and the metallic equivalent to be reduced inside of the aqueous suspension is greater than or equal to 5, preferably greater than 10.

The step of reduction of the metal ions can be carried out by a person skilled in the art according to any known method suitable for the purposes of the present invention.

By way of example, the reduction can be carried out in the presence of a reducing agent such as, e.g., glucose, starch, sucrose, fructose, ascorbic acid or any other natural reducing agent that is compatible with human physiology.

In particular, the concentration of the reducing agent may be comprised between 0.1 and 2.5% (w/w).

The reaction is conducted by stirring for a time of 5-10 minutes the aqueous suspension comprising the chitin nanofibrils and the metal ions. Preferably, it is carried out at a temperature comprised between 20-25° C.

In a preferred embodiment of the invention, the reducing agent is glucose. By way of non-limiting example, the process for the preparation of the suspension may be carried out by using an aqueous solution comprising 2% of nanofibrillar/nanocrystalline chitin and 0.2% of Ag, Cu and/or Bi sulfate or nitrate, and stirring the solution in the presence of glucose at a temperature comprised between about 20-25° C. for a time of about 5-10 minutes.

Suspension Comprising Nanofibrils in a Complex with Metals.

Object of the present invention is also an aqueous suspension comprising the above-mentioned chitin nanofibrils in a complex with one or more metals and a thickener or thickener mixture.

The term “thickener” refers to an agent capable of increasing the density or the viscosity of an aqueous suspension of chitin nanofibrils in a complex or not in a complex with metals. For the purposes of the invention, any agent or mixture of known agents falling within the above definition may be used as thickener in said suspension.

In one embodiment of the invention, said at least one thickener may be selected in the group comprising ethylene polyoxide, polyethylene glycol of different molecular weight, for instance, PEG from 400 to 6000, polylactic acid, glycerol, and polyglycols of different molecular weight or mixtures thereof.

Moreover, the suspension may optionally comprise one or more polymers different from chitin, such as, e.g., chitosan, collagen, gelatin, cellulose, in order to modulate the physico-chemical properties of the material itself.

Preferred polymers different from chitin are polysaccharide polymers. In particular, the presence of a polymer contributes, together with the thickener, to the definition of the biological and mechanical characteristics such as biodegradability, solubility, hydrophily/hydrophoby, degree of deformation, pharmacological activity, for instance the bactericidal, fungicidal, etc., activity of the suspension.

For workability's sake, the chitin nanofibrils are formulated and used in a base solution/suspension containing about 2% (w/w) of nanofibrils in aqueous solution. Therefore, the percent amounts of “chitin nanofibrils” indicated in the present application normally refer to the percent of said base solution/suspension in the liquid mixture of chitin-metals in a liquid medium.

The liquid mixtures normally contain about 30% of said 2% chitin base suspension. Therefore, in absolute value the suspensions of the invention normally contain about 0.6% of chitin nanofibrils. However, different percentages of base suspension can be used for the preparation of the suspensions of the invention. Mixtures containing between 10% and 50% or more (w/w), e.g., 15%, 20%, 25%, 30%, 35%, 40%, 45% of base suspension may equally be used, under certain circumstances, for the purposes of the invention.

Process for the Manufacturing of Biomedical Materials

A process for the manufacturing of biomedical materials from the aqueous suspension of the invention is described hereinafter.

Such process is characterized in that it comprises at least one step of transforming the nanochitin suspension, in a complex with metals, by a technique allowing to deposit and at least partially dry said suspension in a solid or paste-like thin layer.

By “solid or paste-like fine layer” it is meant a sheet, a lamina, a film, a pellicle, a non-woven tissue, etc., both in a solid and semi-solid form, and with a thickness variable needwise. The thickness of said thin layer, regardless of the technique used, ranges in the order of nanometers to millimeters, e.g. from 200 nm to 5 mm, preferably from 0.5 micrometers (microns; μm) to 1 mm, or from 1 μm to 0.5 mm. For instance, the thickness will be of 200 nm, 300 nm, 500 nm, 800 nm, 1 μm, 10 μm, 50 μm, 100 μm, 800 μm, 1 mm, 5 mm.

The suspension used in the manufacturing process can comprise an amount of base suspension (2%) of nanofibrils, in a complex with metal, higher than 5% (v/v), preferably higher than 10% or 20% relative to the total volume of the suspension to be processed.

In one embodiment of the invention, the amount of base suspension of chitin nanofibrils in a complex is of 30% (v/v) of said total suspension.

The suspension may optionally further comprise one or more polymers different from chitin, such as, e.g., collagen, gelatin, cellulose, chitosan.

The transforming of the suspension in a solid or paste-like thin layer can be carried out by the expert in the art according to any technique capable of transforming the suspension in accordance to what indicated herein.

In a first embodiment of the invention, the technique used to obtain materials in form of solid or semi-solid fine layer is electrospinning.

In a second embodiment of the invention, the technique used to obtain materials in form of solid or semi-solid fine layer is casting.

Electrospinning

Electrospinning, also called electrostatic spinning, allows, starting from the above-described thickened suspension, to obtain materials comprised of chitin monofilaments in a complex with metal particles and optionally with other polymers. Such filaments have a sectional diameter smaller than 100 nm and a length of various millimeters or even centimeters.

For this purpose, electrospinning may be carried out by using NS LAB 500S equipment, based on Nanospider Technology.

Advantageously, by using the Nanospider Technology technique, materials are obtained in which the fine layer is in form of non-woven tissue.

In particular, the electrospinning can be carried out at a temperature of 18-30° C., in an environment with a relative humidity between 20-25% and a voltage comprised between 45 and 60 kV.

According to the operation modes adopted, as will be evident to a person skilled in the art, both a material in form of fine monolayer and a material in form of multilayer could be obtained. In fact, by subjecting in sequence to electrospinning suspensions of nanochitin complexed with different metals or with metals containing different polymers, it is possible to obtain multilayer materials in which each layer could have specific chemico-physical and pharmacological characteristics.

Alternatively, by using in sequence a suspension having always the same composition it is possible to obtain a multilayer material in which each layer has the same chemico-physical characteristics.

Casting

Casting is a technique allowing to obtain from the thickened suspension a solid, semi-solid or paste-like fine material in form of film, membrane, pellicle, lamina.

By way of example, the casting could be carried out by distributing on a suitable smooth substrate the suspension of the invention, for instance at 30% of base suspension, so that it has a suitable thickness, e.g. of the order of some micrometers or of some millimeters. Then the distributed suspension is dried, e.g., at a temperature comprised between 20 and 30° C., preferably about 25° C.

The films thus obtained have a thickness preferably as already indicated in the description of the process for the manufacturing of the materials of the invention.

In one embodiment of the invention, the equipment used for the preparation of the matrix is MSK AFA L800.

According to the operation modes adopted, as it will be evident to an expert in the art, both monolayer materials and multilayer materials (with at least two thin layers) could be obtained. In fact, by depositing in sequence, by casting, different suspensions it is possible to obtain multilayer materials with individual layers having alike or different chemico-physical or pharmacological properties.

Following the casting procedure in accordance to what described above, the films consisting of or comprising the complexes of chitin nanofibrils and metals exhibit excellent mechanical properties, measured in terms of maximum tensile stress, Young's modulus and tensile strength higher than those of known films.

Biomedical Material

By “biomedical material” or “material” it is meant a solid or semi-solid thin layer obtainable from a suspension of the invention with any of the above-described processes.

The material is fundamentally comprised of chitin nanofibrils in a complex with metals Ag, Cu and/or Bi.

The material can also comprise one or more polymers different from chitin, like, e.g., chitosan, collagen, gelatin, cellulose.

In a first embodiment such biomedical material is in form of monolayer or multilayer non-woven tissue, obtained by electrospinning.

In a second embodiment the biomedical material is in form of monolayer or multilayer polymer film, obtained by casting.

The material can be used as such as biomedical article, or alternatively used for the manufacturing of more complex biomedical articles.

In one embodiment, the material of the invention can be used as such as medication for damaged tissues.

Biomedical Articles

The material of the present invention can be used for the manufacturing of biomedical articles comprising or consisting of the biomedical material of the invention. Besides the material, the articles can also comprise further components.

By the wording “biomedical article” is meant an article capable of fostering restoration of normal physiology of a tissue, organ, organism, therefore particularly suitable to promote cell proliferation and/or regeneration.

By way of example, such articles may be in form of bandages, gauzes, bands, protection membranes.

In a specific embodiment of the invention, the complex biomedical article comprises at least one layer obtained by casting technique or at least one layer obtained by electrospinning technique.

The articles of the invention can be also multilayer articles comprising plural layers of a same type of material, or plural layers of different types of material of the invention.

By the wording “different types of material” are meant materials obtained with different deposition techniques. Otherwise, with the same wording are meant materials that, though obtained with the same technique, are different in composition, for instance, with reference to the type of metal in a complex with chitin nanofibrils, or to the type of polymer and/or thickener present in the material. Vice versa, by the wording “a single type of material” reference is made to the fact that the solid layer is obtained always by the same technique and always has the same composition.

In a first embodiment, the multilayer biomedical article comprises: at least two layers obtained by casting technique or at least two layers obtained by electrospinning technique according to the process described herein.

In a second embodiment of the invention, the multilayer biomedical article comprises: at least one layer obtained by casting technique and at least one layer obtained by electrospinning technique.

In a further embodiment of the invention, the multilayer article comprises or consists of chitin layers in a complex with metals, alternating with layers based on other polymers.

The biomedical articles described herein are suitable for promoting cell proliferation and/or regeneration, and therefore can be advantageously used as cell growth support or medications and protection in the treatment of wounds, burns, abrasions, cuts, sores, mucosal and skin inflammation. By way of example, such medications can be in form of bandages, plasters, gauzes, bands, protection masks or pellicles.

### EXAMPLES

The following examples are provided for illustration purposes and are not intended to limit the invention as otherwise described herein.

### Example 1

Preparation of the Suspension:

A concentrated aqueous suspension of CN is diluted until obtaining a 2% suspension and supplemented with a 3% Cu sulfate solution until obtaining a suspension with 0.2% sulfate in the suspension. The suspension is thickened by adding, under stirring and at a temperature comprised between 20-25° C., 7% of ethylene polyoxide in powder. The suspension is left under stirring for about 10 minutes, until a homogeneous suspension is obtained.

### Example 2

Preparation of Chitin Nanofibrils (CN)-based Tissue in a Complex with Metals and Ethylene Polyoxide

Electrospinning is carried out by Elmarco's NS LAB 500S equipment under the following conditions and with the following operation steps:
the distance between electrodes is set at 10.5 cm.
the suspension to be electrospun is poured into the holding compartment of the electrode.
electrode rotation rate is set at 2.5 rpm.
voltage is gradually increased until, at about 45 kV, the nanofibres or nanofilaments begin to form.
the substrate spooling system is activated and electrospinning is carried on until the suspension lasts.
sample is left to dry for about 1 hour, then collected.

### Example 3

Protocol for Obtainment of Films According to the Casting Technique.

Casting is carried out by Elmarco's MSK afa 1800 equipment under the following conditions and with the following operation steps:
connect machinery with a 220/110 V power supply.
activate by pressing “reset” button.
position the suspension of the invention on the specially provided tempered glass dish.
calibrate film applier (that provided is a 250-nm one) according to instructions reported in the operation manual.
assemble “pusher2” as illustrated on the manual.
position it on the suspension.
activate machinery.
adjust position so as to have the desired thickness.
adjust flow rate.
press “run” to initiate casting process.
once casting has ended, collect excess material in the specially provided waste tray.
let dry the film.
turn off machinery and collect obtained film.

### Example 4.

Composition of the Suspension: (chitosan/chitin in a Complex with Silver)/PEG-600=(70/30)/50% by Weight

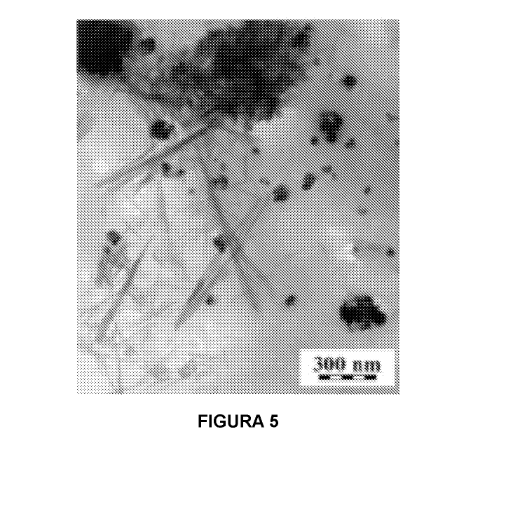

1.75 g of chitosan in powder were dispersed in 130 ml distilled water in a 500 ml round-bottom flask placed on a magnetic stirrer. Then, 0.5 ml of 99% acetic acid were added. The suspension was stirred at room temperature for 3 hours. The homogeneous chitosan solution was filtered through a #2 glass filter to remove any mechanical particles and additives. Then, the filter was washed with 130 ml of distilled water and the filtrates (250 ml, pH=4,63) accurately mixed. In order to prepare a suspension with a composition of chitosan/CN-Ag=70/30% by weight, to the chitosan solution a defined amount of emulsion of chitin nanofibrils in a complex with silver (CN-Ag) (concentration: 43.9 mg/ml; produced by Mavi Sud S.r.l., Italy) was added under stirring (FIG. 5). Then, PEG 600 thickener (50% by weight from the total weight of the dry matter in the suspension) was added and the mixer vigorously stirred for 0.5 hours. The concentration of acetic acid and of chitosan in the suspension was respectively of about 0.7% by weight and 0.2% by weight. Lastly, excess water (80 ml) and air bubbles were removed from the suspension by evaporation under vacuum. Water bath temperature was of 40±0.1° C. The suspension was poured on glass slides hydrophobized beforehand with dimethyl dimethoxysilane in order to more easily remove the dried chitosan/CN-Ag nanocomposite films from the support glass. Suspension layer height was of 1-1.5 mm. After having dried the suspension under stirred air flow at 25±1° C., the chitosan/CN-Ag films were removed from the supports and tested without any additional treatment. Chitosan/CN-Ag dry film thickness is of 40±10 micrometers.

TABLE 1
Mechanical characteristics of the chitosan/CN—Ag film
thickened with PEG-600.
Supports-glass dishes with silane (contact angle: 103.8 ± 0.7)

| | | Maximum | | |
|---|---|---|---|---|
| | Composition | tensile | Tensile | Young's |
| | (CS/CN—Ag)/PEG-600, | stress | strength | moduli |
| Sample | % by weight | MPa | % | MPa |
| 1 | (70/30)/50 | 36.8 ± 4.8 | 18.1 ± 3.1 | 852 ± 166 |
| 2 | ″ | 42.5 ± 5.7 | 20.7 ± 1.7 | 842 ± 143 |
| 3 | ″ | 50.5 ± 2.6 | 22.7 ± 2.9 | 905 ± 191 |
| 4 | ″ | 49 ± 5.2 | 17.2 ± 3.8 | 134 ± 374 |

### Example 5

Composition of the Suspension: (chitosan/CN-Ag)/glycerol

1.75 g of chitosan in powder were dispersed in 200 ml of distilled water in a 500-m1 round-bottom flask placed on a magnetic stirrer. Then, 0.5 ml of 99% acetic acid were added. The suspension was stirred for 3 hours to temperature. The homogeneous chitosan solution was filtered through a #2 glass filter to remove any mechanical particles and additives. Then, the filter was washed with 60 ml of distilled water and the filtrates (250 ml, pH=4,65) accurately mixed. In order to prepare the suspensions with the desired compositions (Table 2), a definite amount of chitin nanofibrils emulsion in a complex with silver (CN-Ag) (produced by Mavi Sud S.r.l., Italy) with a concentration of 22.9 mg/ml was added to the chitosan solution under stirring.

Then, the thickener glycerol (20, 40 or 50% by weight, relative to the weight of the dried matter of the suspension) was added, and the mixture was vigorously stirred for 0.5 hours. Finally, excess water (100-110 ml) and water bubbles were removed from the suspension by evaporation under vacuum. Water bath temperature was of 40±0.1° C. The suspension was poured on glass slides hydrophobized beforehand with dimethyl dimethoxysilane. The height of the suspension layer was 1-1.5 mm. After having dried the suspension under stirred-air flow at 25±1° C., the chitosan/CN-Ag films were removed from the supports and tested without any additional treatment. Dry chitosan/CN-Ag film thickness was in the range of 50-60 micrometers.

TABLE 2
Mechanical characteristics of the chitosan/CN—Ag film thickened
with glycerol.
Supports-glass dishes with silane (contact angle: 103.8 ± 0.7)

| | Chitosan/CN—Ag/ | Maximum | | | |
|---|---|---|---|---|---|
| | glycerol | tensile | Ultimate | Young's | Film |
| | composition | stress | strength | moduli | contact |
| Sample | % by weight | MPa | % | MPa | angle |
| 5 | (80/20)/20 | 86.5 ± 4.9 | 8.9 ± 2.5 | 4086 ± 410 | 95.8 ± 2.2 |
| 6 | (60/40)/20 | 92.1 ± 5.5 | 10.4 ± 2 | 4161 ± 475 | 9.80 ± 2.1 |
| 7 | (40/60)/40 | 61.6 ± 4.4 | 9.3 ± 2 | 3093 ± 780 | 104.0 ± 1.7 |
| 8 | (40/60)/50 | 59.9 ± 7 | 8.8 ± 3 | 3230 ± 700 | 97.2 ± 2.0 |
| 9 | (20/80)/50 | 37 ± 2.8 | 6.1 ± 1.8 | 2582 ± 466 | 92.3 ± 5.9 |
| 10* | (40/60)/50 | 21.6 ± 1.8 | 19.5 ± 2.5 | 211 ± 35 | 107.4 ± 1.5 |

| | | | | |
|---|---|---|---|---|
| Cuticell-a | 55 ± 31 | 1.2 ± 0.5 | 7000 ± 1600 | — |
| Cuticell-b | 68.5 ± 22 | 1.3 ± 0.7 | 9350 ± 200 | |
| Jaloskin-a | 59 ± 15 | 2.50.9 | 3950 ± 240 | — |
| Jaloskin-b | 75 ± 9 | 2.7 ± 0.6 | 4215 ± 235 | |

*In the preparation of Sample 10, instead of the emulsion of nanofibrillar chitin in a complex with silver (CN—Ag), the thin aqueous dispersion of chitin microparticles in a complex with Ag is used.

### Example 6

Composition of the Suspension: (chitosan/CN-Ag)/Polyglycerols or PEG-600

0.8 g of chitosan in powder were dispersed in 50 ml of distilled water in a 250-ml round-bottom flask placed on a magnetic stirrer. Then, 0.8 ml of 99% acetic acid were added. In order to obtain complete dissolution of chitosan, the mixture (pH=4.0) was stirred for 2 hours at a temperature of 50° C. The viscous and homogeneous solution of chitosan was mixed under intense stirring with the definite amount of CN-Ag emulsion (produced by Mavi Sud S.r.l., Italy) (Table 3) with an emulsion concentration of 16.0 mg/ml. Then, glycerol, polyglycerol-2, polyglycerol-3 or polyglycerol-4 (Solvay Chemicals International S.AS., Belio) thickener was added (20 or 30% by weight of the total weight of the dry matter in the suspension) and the mixture vigorously stirred for 0.5 hours. The final concentration of chitosan and acetic acid in the suspension was of about 2% by weight. Lastly, excess water (10-11 ml) and air bubbles were removed from the suspension by evaporation under vacuum. Water bath temperature was of 40±0.1° C. The suspension was subjected to casting on Dura Lar films (Grafix, USA) using MSK AFA L800 equipment (MTI Corp.; USA). The height and width of the opening of the doctor blade used for the casting of the suspension were respectively of 0.075 and 25 cm. After drying, the suspension under air flow was stirred at 25±1° C., the chitosan/CN-Ag films were removed from and characterized without any additional treatment. The thickness of the chitosan/CN-Ag film was in the range of 40-50 micrometers.

TABLE 3
Mechanical characteristics of the chitosan/CN—Ag/polyglycerols film =
(85/15)/20 or 30% by weight.
Support: DUra Lar film (contact angle-76.1 ± 0.9)

| | | Thickener | Maximum | Ultimate | Young's |
|---|---|---|---|---|---|
| Sam- | | content % | tensile stress | strength | moduli |
| ple | Thickener | by weight | MPa | % | MPa |
| 11 | chitosan/ | 0 | 26.8 ± 3.5 | 6.1 ± . | 2244 ± 180 |
| | CN—Ag | | | | |
| | (85/15) | | | | |
| 12 | Glycerol | 20 | 4. ± 5 | 20.4 ± 4 | 1390 ± 600 |
| 13 | ″ | 30 | 35.4 ± 4 | 27.6 ± 4 | 300 ± 50 |
| 14 | Diaglycerol | 20 | 48.6 ± 8 | 25.4 ± 4.7 | 1835 ± 177 |
| 15 | ″ | 30 | 36.7 ± 5 | 32.5 ± 5.7 | 700 ± 65 |
| 16 | Triglycerol | 20 | 33.5 ± 6 | 26.7 ± 8 | 1390 ± 100 |
| 17 | ″ | 30 | 25 ± 5 | 35 ± 6 | 379 ± 47 |
| 18 | Tetraglycerol | 20 | 41.4 ± 4.9 | 13.7 ± 5 | 2320 ± 110 |
| 19 | ″ | 30 | 30 ± 4.5 | 17 ± 5 | 1380 ± 50 |
| 20 | ″ | 40 | 28.6 ± 6 | 31.6 ± 7 | 370 ± 57 |
| 21 | Peg600 | 20 | 36.8 ± 2.2 | 26 ± 5 | 1620 ± 200 |
| 22 | ″ | 30 | 37.6 ± 4.9 | 25 ± 4.4 | 1730 ± 350 |
| 23 | ″ | 40 | 22.3 ± 2.6 | 21 ± 3 | 895 ± 64 |
| 24 | ″ | 40 | 23.1 ± 3.3 | 34.5 ± 7.5 | 870 ± 60 |
| 25 | ″ | 50 | 20.4 ± 2.6 | 24 ± 5 | 765 ± 80 |
| 26* | ″ | 50 | 40.45 ± 3.8 | 18 ± 4 | 993 ± 256 |

*Chitosan/CN—Ag = 70/30% by weight (produced by Mavi Sud S.r.l., Italy)

### Example 7

Composition of the Suspension: (chitosan/CN-Ag)/glycerol=(85/15) 30% by weight.

0.8 g of chitosan-2 in powder were dispersed in 30 ml of distilled water in a 250-m1 round-bottom flask placed on a magnetic stirrer. Then, 0.4 ml of 99% acetic acid were added. The mixture (pH=4.32) was stirred for 12 hours at room temperature. In order to prepare the suspension with a composition: (chitosan/CN-Ag/glycerol=(85/15) 30% by weight), the homogeneous solution of chitosan was mixed with a definite amount of CN-Ag emulsion (produced by Mavi Sud S.r.l., Italy) with a concentration of 16.0 mg/ml of the emulsion (pH=5.7) and glycerol. The final concentration of chitosan and acetic acid in the suspension was of about 2% by weight. Lastly, excess air bubbles was removed from the suspension by evaporation under vacuum. The suspension was subjected to casting on PET film (Grafix, USA) using MSK AFA L800 equipment (MTI Corp.; USA). The height and width of the opening of the doctor blade used for the casting of the suspension were respectively of 0.075 and 25 cm.

Film drying was carried out under stirred air flow at 40±45° C. or with infrared irradiation at 50-55° C. After removal of the chitosan film from the supports, they were used without any additional treatment. Film thickness was 30-35 micrometers.

TABLE 4
Mechanical characteristics of the chitosan/CN—Ag/glycerol film =
(85/15)/20 30% by weight.
Support: PET (contact angle-77.4 ± 1.9)

| | | Maximum tensile | Ultimate | Young's |
|---|---|---|---|---|
| | | stress | strength | moduli |
| Sample | Drying conditions | MPa | % | MPa |
| 27 | Infrared irradiation at | 43.5 ± 4.8 | 15 ± 3.8 | 2257 ± 392 |
| | 50° C. for 1 hour | | | |
| 28 | Infrared irradiation at 50-55° C. for | 35.9 ± 5.1 | 18 ± 4.5 | 1957 ± 202 |
| | 0.5 hours | | | |
| 29 | Infrared irradiation at 50-60° C. for | 45 ± 4.5 | 12 ± 3 | 2495 ± 380 |
| | 0.5 hours | | | |
| 30 | Stirred air flow, | 25 ± 3.7 | 12.6 ± 2.8 | 1462 ± 413 |
| | 40-50° C., for 2 hours | | | |

### Example 8

Composition of the Suspension: (chitosan/CN-Ag)/diglycerol or triglycerol.

0.8 g chitosan (CS) in powder were dispersed in 30 ml of distilled water in a 250-ml round-bottom flask placed on a magnetic stirrer. Then, 0.8 ml of 99% acetic acid were added. To obtain complete dissolution of the chitosan, the mixture (pH=4.0) was stirred for 2 hours at a 50° C. temperature. In order to prepare the suspension with the composition (chitosan/CN-Ag)/poliglycerol=(85/15)/30% by weight or (65/35)/30% by weight, the homogeneous solution of chitosan was mixed with a defined amount of CN-Ag emulsion (produced by Mavi Sud S.r.l., Italy) with a 16.0 mg/ml concentration of the emulsion (pH=5.7) and diglycerol or triglycerol. The final concentration of chitosan and of acetic acid in the suspension was, respectively, of about 2.2, 1.8 or 1.5% by weight. Lastly, air bubbles were removed from the suspension by evaporation under vacuum. The suspension was subjected to casting on PET film (Grafix, USA) using an MSK AFA L800 equipment (MTI Corp.; USA). Height and width of the opening of the doctor blade used for the casting of the suspension were respectively of 0.075 and 25 cm. Film drying was carried out under stirred air flow at 25±1° C. Chitosan/CN-Ag films, after removal from the supports, were used without any additional treatment. Film thickness was of 30-35 micrometers.

TABLE 5
Mechanical characteristics of the chitosan/CN—Ag film with diglycerol or
triglycerol.
Support: Dura Lar film (contact angle-76.1 ± 0.9)

| | | CS/CN—Ag/ | | | |
|---|---|---|---|---|---|
| | | polyglycerol | Maximum | Ultimate | |
| | | content | tensile stress | strength | Young's moduli |
| Sample | Thickener | % by weight | MPa | % | MPa |
| 31 | Diglycerol | (85/15)/30 | 33 ± 7 | 32.3 ± 8.5. | 178 ± 53 |
| 32 | ″ | (75/25)/30 | 34.6 ± 6.2 | 35.3 ± 5.9 | 135 ± 20 |
| 33 | ″ | (65/35)/30 | 30 ± 6.8 | 35 ± 7.5 | 125 ± 17 |
| 34 | Triglycerol | (85/15)/30 | 29.3 ± 3.9 | 46.9 ± 5.6 | 72.8 ± 6.5 |
| 35 | ″ | (75/25)/30 | 24 ± 6.5 | 37.5 ± 7.2 | 89.5 ± 10 |
| 36 | ″ | (65/35)/30 | 23.4 ± 5.8 | 41.8 ± 7.9 | 71.3 ± 11.2 |

### Example 9

The bacteriostatic/bactericidal activity of non-woven tissues (NT) obtained by electrospinning, consisting of chitin nanofibrils (CN) in a complex with silver (Ag), copper (Cu) and/or bismuth (Bi) and Chitosan (CS) was assessed with respect to non-woven tissues consisting of chitin nanofibrils (CN) and Chitosan (CS), after 18 hours of incubation at 36° C. on agar containing a bacterial culture obtained from non-sterilized cutaneous tissue.

TABLE 6

| Sample | Bacterial growth (CFU/g) |
|---|---|
| Agar + cutaneous tissue (CB) | 10⁷ |
| (control). | |
| Agar + CB + NT(CS/CN) | 10⁵ |
| Agar + CB + NT(CS/CN—Ag) | 10³ |
| Agar + CB + NT(CS/CN—Cu) | 10³ |
| Agar + CB + NT(CS/CN—Bi) | 10³ |
| Agar + CB + NT(CS/CN—Ag—Cu—Bi) | 10^{1.7} |

CB = non-sterilized cutaneous tissue
NT(CS—CN) = non-woven tissue consisting of Chitin and chitosan

### Example 10

The bacteriostatic/bactericidal and fungicidal/fungistatic activity on C. albicans, St. aureus and E. coli strains of the non-woven tissues and of the films comprising chitin nanofibrils (CN) in a complex with silver (Ag), copper (Cu) and/or bismuth (Bi) was evaluated with respect to reference samples. Standard applied for antimicrobial activity: SNV 195920 with minor modifications. In particular, the assessed samples were:
1) CN-based non-woven tissue (comparison standard);
2) Ag ions-complexed CN-based film;
3) Ag+Cu+Bi ions-complexed CN-based film;
4) Ag ions-complexed CN-based non-woven tissue;
5) Ag+Cu+Bi ions-complexed CN-based non-woven tissue.

Results obtained on the growth of C. albicans, St. aureus and E. coli strains are schematized in Table 7 below.

TABLE 7

| | C. albicans | St. aureus | E. coli |
|---|---|---|---|
| SAMPLES | ATCC90028 | ATCC6538 | LAC- |
| 1 | insufficient | insufficient | insufficient |
| 2 | good | good | good |
| 3 | insufficient | good | good |
| 4 | limited | good | good |
| 5 | insufficient | good | limited |

Insufficient = Complete growth or ≦50% reduction
Limited = ≧70-90% inhibition of growth
Good = growth absent.

The invention has hereto been described with reference to embodiments thereof. As it will be apparent to a person skilled in the art from what detailed, also further embodiments falling within the concept of the same invention could be provided which, therefore, will have to be construed as encompassed by the protective scope defined by the claims of the present application.

1. An aqueous suspension comprising at least one thickener and chitin nanofibrils in a complex with at least one metal selected from the group consisting of Ag, Cu and Bi.

2. The suspension according to claim 1, wherein said thickener is selected from the group consisting of ethylene polyoxide, polyethylene glycol, polylactic acid, glycerol, and polyglycols.

3. A process for the preparation of the suspension according to claim 1, comprising at least one step of reducing ions of at least one metal selected from the group consisting of Ag, Cu and Bi in an aqueous suspension which comprises chitin nanofibrils.

4. The process according to claim 3, wherein the concentration ratio between said chitin nanofibrils and said ions in the aqueous suspension is greater than or equal to 5, preferably greater than or equal to 10.

5. The process according to claim 3, wherein said step of reduction is carried out in the presence of glucose.

6. The process according to claim 3, wherein said step of reduction is carried out at a temperature comprised between 20-25° C. and for a time comprised between 5-10 minutes.

7. A biomedical material in form of non-woven tissue, film, sheet, pellicle obtainable by a process of casting or electrospinning of an aqueous suspension comprising at least one thickener and chitin nanofibrils in a complex with at least one metal selected from the group consisting of Ag, Cu and Bi.

8. The biomedical material according to claim 7, wherein said thickener is selected from the group consisting of ethylene polyoxide, polyethylene glycol, polylactic acid, glycerol, and polyglycols.

9. The biomedical material according to claim 7, wherein said aqueous suspension comprises one or more additional polymers different from chitin.

10. The biomedical material according to claim 7, wherein the electrospinning is carried out at a temperature comprised between 18 and 30° C., in an environment with a relative humidity between 20-25% and a voltage comprised between 45 and 60 kV.

11. The biomedical material according to claim 7, wherein the casting is carried out at a temperature comprised between 20° C.-30° C. or between 20° C.-25° C.

12. A process for the manufacturing of the biomedical material according to claim 7, comprising a step of transforming a suspension comprising at least one thickener and chitin nanofibrils in a complex with at least one metal selected from the group consisting of Ag, Cu and Bi in a solid, semi-solid,. or paste-like fine layer by electrospinning or casting.

13. The process according to claim 12, wherein the electrospinning is carried out at a temperature comprised between 18 and 30° C., in an environment with a relative humidity between 20-25% and a voltage comprised between 45 and 60 kV.

14. The process according to claim 12, wherein the casting is carried out at a temperature comprised between 20° C. and 30° C. and optionally followed by thin layer drying.

15. A biomedical article comprising or consisting of the biomedical material according to claim 7.

16. The biomedical article according to claim 15, in form of monolayer or multilayer.

17. The biomedical article according to claim 16, comprising:
at least one layer consisting of or comprising the material in form of non-woven tissue and/or
at least one layer consisting of or comprising the material in form of film, sheet or pellicle.

18. The biomedical material according to claim 7 or a biomedical article comprising or consisting of said biomedical material in form of bandage, plaster, gauze, band, protection mask or pellicles, or cell growth support.

19. A method of treatment of wounds, burns, abrasions, cuts, sores, mucosal or skin inflammation, comprising administering the biomedical material according to claim 7 or a biomedical article comprising or consisting of said biomedical material to a subject in need thereof.
